SEQUENCE LISTING (1 sequence)

SEQ ID NO: 1 - protein, 148 aa, synthetic construct
MGSDKIHHHHHHENLYFQGGCGGSDLGKKLLEAARAGQDDEVRILMANGADVNAYDDNGVTPLHLAAFLGHLEIVEVLLKYGADVNAADSWGTTPLHLAATWGHLEIVEVLLKHGADVNAQDKFGKTAFDISIDNGNEDLAEILQKLN

(19) United States
(12) Patent Application Publication
(10) Pub. No.: US 2024/0424116 A1
(43) Pub. Date: Dec. 26, 2024

(54) Pegylated-AU(III) Reagents for Rapid Cysteine S-Arylation of Biomolecules

(71) Applicant: The Regents of the University of California, Oakland, CA (US)

(72) Inventors: Heather D. Maynard, Los Angeles, CA (US); Alexander Spokoyny, Woodland Hills, CA (US); Hayden Montgomery Aytac, Oakland, CA (US); Marco Messina, Los Angeles, CA (US); Evan Doud, Oakland, CA (US)

(21) Appl. No.: 18/336,776

(22) Filed: Jun. 16, 2023

(51) Int. Cl. A61K 47/64; A61K 33/38; A61K 47/52

(52) U.S. Cl. CPC A61K 47/64; A61K 33/38; A61K 47/52

(57) ABSTRACT

Compositions and methods for cysteine arylation of polypeptides using Au(III) organometallic complexes are disclosed herein. The bioconjugation reactions proceed rapidly at ambient temperature in various buffers. The compositions and methods enable the synthesis of PEGylated polypeptide conjugates with selectivity and high efficiency, allowing facile modification of complex biomolecules.

### STATEMENT REGARDING FEDERALLY SPONSORED RESEARCH

This invention was made with government support under OD028491, and GM124746 awarded by the National Institutes of Health, and 2003946, and 0707424 awarded by the National Science Foundation. The government has certain rights in the invention.

### REFERENCE TO A SEQUENCE LISTING

A Sequence Listing accompanies this application and is submitted as an ASCII text file of the sequence listing named “790482_00328_Sequence_Listing.xml” which is 1,921 bytes in size and was created on Jun. 15, 2023. The sequence listing is electronically submitted via EFS-Web with the application and is incorporated herein by reference in its entirety.

### FIELD OF THE INVENTION

The disclosed technology is generally directed to modification of macromolecules. More particularly the technology is directed to modification of macromolecules with polyethylene glycol.

### BACKGROUND OF THE INVENTION

Protein and peptide therapeutics represent a large class of treatments for a plethora of diseases, with more than 100 therapeutics garnering approval by the Food and Drug Administration (FDA) for clinical use.¹⁻³ Conjugation of synthetic polymers to these biomacromolecule therapeutics significantly increases both the stability and in vivo lifetime of the drug, making the application of proteins and peptides as disease treatments more feasible and effective.⁴ Currently, polyethylene glycol (PEG) comprises the polymer component of all of the 27 protein-polymer conjugates that are approved by the FDA. Therefore, the pharmacokinetics and immunogenicity of PEG conjugates are well established, and the ability of the polymer to stabilize therapeutics has been shown extensively.⁵⁻⁷

Bioconjugation techniques for the efficient synthesis of PEGylated-biomacromolecular conjugates generally necessitate a coupling strategy with extremely rapid kinetics and high chemoselectivity to overcome the steric hindrance and low target functional group concentration inherent to these systems. Examples of these types of bioconjugation strategies are numerous; however, most require large excess of polymer reagents, often 10 equivalents or more.⁸⁻¹⁴ This large excess of reagent not only exhibits poor atom economy, but it also necessitates complicated purification strategies to separate multiple macromolecules from one another. Additionally elevated reaction temperatures are occasionally employed to achieve near quantitative conversion, which can be detrimental to the structure and function of more delicate biomolecules.^{9, 15, 16} Furthermore, these modifications can also perturb the natural structure and activity of the biomolecule.

In contrast, classical canonical bioconjugation techniques often utilize nucleophilic amino acid residues, particularly lysine and cysteine, to achieve efficient conjugation.¹⁸⁻²⁴ However, modification of lysine residues often provides poor residue selectivity and can affect the solubility, structure, and stability of the resulting biomolecule conjugate. To circumvent these challenges, free cysteines are often employed as nucleophilic amino acid residues for bioconjugation, due to low abundance and ease of engineering into proteins. Further benefits of the thiol functionality of cysteine include its unique chemical characteristics, such as its ionizability and soft nucleophilicity. However, traditional cysteine conjugations, such as maleimide conjugations and disulfide exchanges, forge bonds that are generally considered reversible in biologically relevant media.²⁵⁻²⁹ While this can be advantageous in some cases where release of the protein is desired, in others, more stable bonds are preferred. Certain pH or redox conditions found in the body can undesirably cleave labile bonds, causing decomposition, clearance, toxic effects or an otherwise negative impact on pharmacokinetic properties.^{26, 31-33}

Cysteine S-arylation has emerged as a strategy toward more stable heteroatom-C(sp2) linkages that can help overcome the existing limitations in cysteine bioconjugation.^{34, 35} Transition metal complexes for arylation of biomolecules have been shown to exhibit high functional group tolerance, chemoselectivity and rapid reaction kinetics to achieve efficient synthesis of bioconjugates.^{34, 36, 37} A Au(III) organometallic cysteine S-arylation approach in which small molecule Au(III) aryl reagents forge stable, S—C(sp2) bonds rapidly and selectively with cysteine thiols via reductive elimination has been disclosed.^{36, 38-42} The relative thiophilicity of Au(III), as well as the reluctance of the Au(I) byproduct to undergo oxidative addition provided the benefit of minimal background reactivity with the many reactive substrates present in a biological setting.

There remains a need for reagents and methods of efficiently PEGylating sensitive macromolecules in high yield to avoid high temperatures that can affect the macromolecule structure and function and to avoid complicated purification schemes for separating excess reagents from the desired product.

### SUMMARY

Disclosed herein are compositions and methods for Au(III)S-arylation of polypeptides. In particular, the synthesis of poly(ethylene glycol) monomethyl ether (mPEG)-protein conjugates is described. Organometallic Au(III) reagents were synthesized and shown to react with a cysteine thiol on a biomolecule to form covalent S-aryl linkages and mPEG-biomolecule conjugates. Notably, low polymer reagent loadings were used to achieve near quantitative conversion at room temperature in one minute due to the rapid kinetics and high chemoselectivity of this approach.

An aspect of the technology provides for compositions for Au(III)S-arylation of polypeptides. The composition may comprise a polypeptide having at least one cysteine residue and a gold(III) aryl complex. The gold(III) aryl complex comprises a polyethylene glycol group having an average molecular weight greater than 1 kDa attached to the aryl group. Suitably the composition may have a molar ratio of the gold(III) aryl complex to the polypeptide of less than 5. In some instances the molar ratio is between 1 and 2. In the compositions described herein, the polypeptide may have a molecular weight greater than 10 kDa.

Another aspect of the technology provides for methods for preparing cysteine-aryl conjugated polypeptide. The method may comprise contacting any of the gold(III) aryl complexes described herein with a polypeptide under conditions sufficient to prepare the cysteine-aryl conjugated polypeptide. Suitably, the method may be performed rapidly under mild conditions. For example, conjugation may be performed in minutes under mild conditions, such as at a temperature less than 30° C., and achieve high conversion efficiency.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows a chemical reaction scheme for the synthesis of mPEG-aryl iodide reagents, according to an aspect of the disclosure herein.

FIG. 2 shows a chemical reaction scheme for the synthesis of mPEG-Au(III) reagents, according to an aspect of the disclosure herein. The percent yields are given for three mPEG variants of molecular weight 2 kDa (2K: 76%), 5 kDa (5K: 80%), and 10 kDa (10K:86%).

FIG. 3 is a synthetic scheme illustrating the modification of a DARPin protein by mPEG-Au(III) reagent leading to the formation of a cysteine-functionalized conjugate, in accordance with the present disclosure.

FIG. 4 is a photograph of an SDS-PAGE gel of 2 kDa, 5 kDa, and 10 kDa PEG-DARPin conjugates.

FIG. 5 is a photograph of an SDS-PAGE gel of crude 5 kDa Au(III) PEG-DARPin conjugates synthesized in 20 mM Tris, 150 mM NaCl buffer, pH 7.5 for 1 minute, utilizing 1, 1.2, 1.5, and 2 equivalents of 5 kDa mPEG Au(III) reagents.

FIG. 6 is a photograph of an SDS-PAGE gel of crude 10 kDa Au(III) PEG-DARPin conjugates synthesized in 100 mM citrate buffer, pH 5.5 after one and 15 minutes at 25° C.

FIG. 7 is a photograph of an SDS-PAGE gel of crude 5 kDa and 10 kDa Au(III) PEG-DARPin conjugates synthesized in 20 mM Tris, 150 mM NaCl buffer, pH 7.5 at 70 μM (5 kDa) and 7 μM (10 kDa) and at both 25° C. and 4° C.

FIG. 8 is a photograph of an SDS-PAGE gel of crude 2 kDa Au(III) PEG-DARPin and 5 kDa maleimide PEG-DARPin conjugates synthesized in 20 mM Tris, 150 mM NaCl buffer, pH 7.5 at 70 μM after one and 15 minutes at 25° C.

FIG. 9 a) A circular dichroism (CD) spectrum of native DARPin and DARPin conjugate at 25° C. showing no significant difference in helicity. b) A CD thermal denaturation curves of native DARPin and DARPin conjugate showing no significant difference in melting temperature between 22-98° C.

FIG. 10 a) MALDI TOF spectra of 2, 5 and 10 kDa (left to right) mPEG Au(III) reagents.

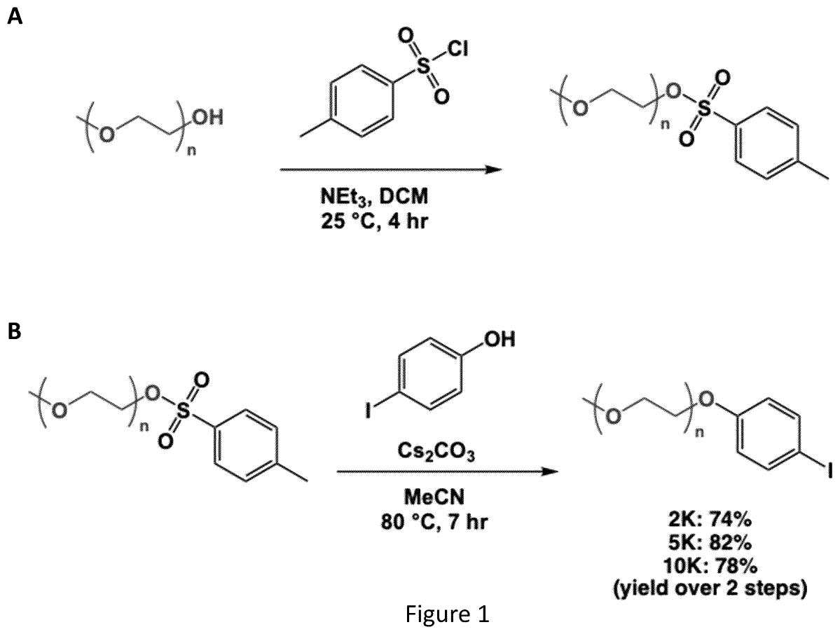

### DETAILED DESCRIPTION OF THE INVENTION

Conjugation of PEG polymers improves the stability and bioavailability of biomacromolecule therapeutics, however bioconjugation techniques for efficient synthesis of PEGylated biomacromolecules present significant challenges. Current methods typically use an excess of polymer reagents resulting in complicated purification strategies to separate the desired product from the mixture of all possible substitution products, starting materials and excess reagents. Current methods also use elevated reaction temperatures which can partially denature or otherwise damage the biomacromolecule to obtain the desired product. Disclosed herein is a gold(III) organometallic complex and a cysteine S-arylation method for efficient PEGylation of a macromolecule using the gold(III) organometallic complex. Notably, rapid reaction rates associated with gold(III) organometallic complex S-arylation allows for the use of near equimolar amounts of polymer reagent and extremely mild reaction conditions can achieve near quantitative conversion in a minute.

Disclosed herein is a composition comprising a polypeptide having at least one residue having a free thiol (e.g., cysteine) and a gold(III) aryl complex. The polypeptide can be a protein or fragment thereof, for example an enzyme, hormone, structural protein, storage protein, contractile protein, an antibody, or an immunoglobulin. The polypeptide can comprise a multiplicity of amino acids residues. Polypeptides may have 10, 50, 100, 150, 200, or 250 or more amino acid residues. For example, polypeptides may have between 10 and 3000 or 50 and 2000, amino acid residues. The size of polypeptides may also be characterized by their mass. Suitably, the polypeptides have a mass of 10, 15, 20, 25, 30, 35, 40, 45, or 50 kDa or more. For example, polypeptides may have a mass between 10 and 1,000, 15 and 500, 20 and 250 or 25 and 100 kDa. The polypeptide can include an unnatural alkyl thiol or a synthetically appended alkyl thiol. The polypeptide can be natural, derived from bioengineering methods, or the polypeptide can be synthetic. In one example, the polypeptide is a designed ankyrin repeat protein (DARPin) having a sequence as given by SEQ. ID NO. 1.

According to an aspect, the composition further includes a gold(III) aryl complex. The gold(III) aryl complex comprises a gold(III) ion with a ligand set that stabilizes the gold(III) complex. The ligand set can be a 2-(di(alkyl)phosphino)-N,N-dimethylaniline, such as shown in exemplary Scheme 1 where R¹ is a cycloalkyl or an alkyl. The cycloalkyl group may be monocyclic, bicyclic, tricylic, or polycyclic. Au complexes can be formed as shown in Scheme 1 with any combination of ligand sets and PEG-substituted aryl groups.

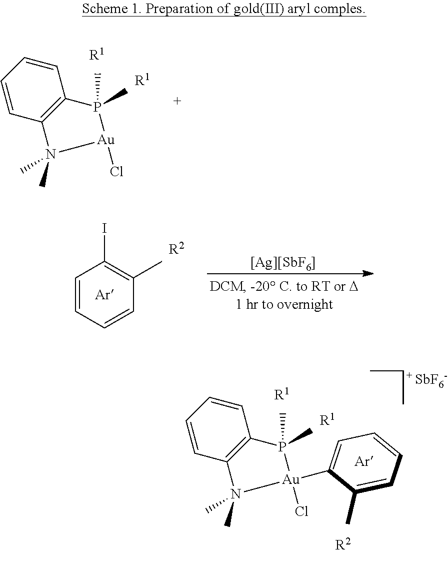

Exemplary R¹ include adamantanyl, cyclohexyl, or tert-butyl groups. In one specific example, the ligand set can be 2-(di(adamantan-1-yl)phosphino)-N,N-dimethylaniline, L1 (Me-DalPhos, CAS No. 1219080-77-9). In still another example, the ligand set can be [2-(di-tert-butylphosphino)-N,N-dimethylaniline, L2 (CAS No. 415941-58-1). In yet another example, the ligand set can be 2-(dicyclohexylphosphino)-N,N-dimethylaniline, L3. In another example, the ligand set can be a 2-(di(alkyl)phosphino)phenyl]morpholine, such as N-[2-(di-1-adamantylphosphino)phenyl]morpholine (Mor-DalPhos; CAS No. 1237588-12-3).

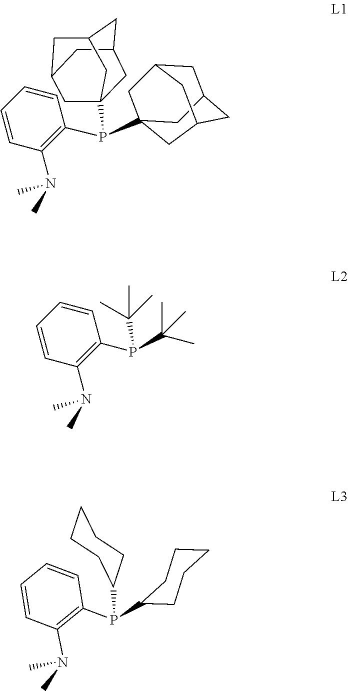

Exemplary gold(III) aryl complexes include, without limitation,

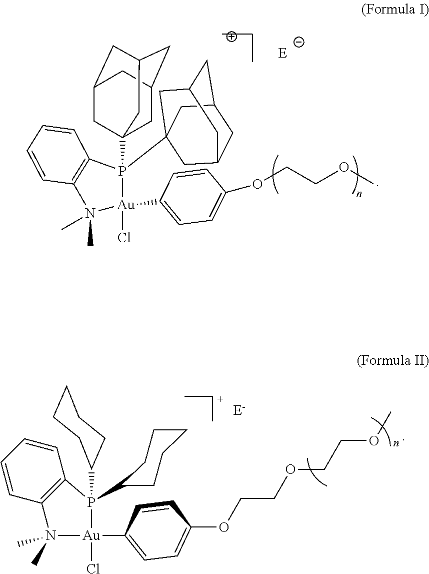

The gold(III) aryl complex can carry a positive charge such that it is associated with a counterion E⁻. The counterion E⁻ can be any anion suitable for use with biomacromolecules, including but not limited to a halide, acetate, formate, carbonate, nitrate, sulfate, phosphate, hexafluorophosphate, tetrafluoroborate, or hexafluoroantimonate.

The aryl group of the of gold(III) complex can be bonded to the gold(III) ion by a gold-carbon bond. The aryl group may be a phenylene or another aryl or heteroaryl group.

The aryl group includes a PEG group attached thereto. PEG is recognized as comprising a oligomeric or polymeric divalent group of formula —(CH₂CH₂O)ₙ— where n is an integer. The PEG group can be positioned at the meta or para position relative to the gold-carbon bond. The PEG group can have an average molecular weight greater than 1 kDa. In another example, the PEG group can have an average molecular weight greater than 2 kDa. In a further example, the PEG group can have an average molecular weight greater than 10 kDa. Additionally, and alternatively, mixed PEG reagents can be used that include a range of molecular weights. The terminus of the PEG group may be varied depending on application. For example, he PEG group can have a methoxy terminus (mPEG). For other examples, the PEG group can a functional terminus conjugated directly or indirectly to a functional group. The functional group can be, for example, a lipid, a peptide, a reactive linker, a nucleic acid, or a small molecule, for example, biotin, adamantane, or pyrene. The PEG group can have a branched structure or be a hyperbranched dendrimer.

The composition can have the gold(III) aryl complex and the polypeptide combined in amounts having a molar ratio. According to an aspect, the molar ratio of gold(III) aryl complex to the polypeptide can be less than 5, 4, 3, or 2 and/or more than 0.1, 0.5, or 1. In another example, the molar ratio of the gold(III) aryl complex to the polypeptide can be between 0.1 and 5, 0.5 and 4, 1 and 3, or 1 and 2.

The concentration of the polypeptide in the composition is dependent on the solubility of the polypeptide. The composition can include at least 1 μM of polypeptide. In some examples, the composition can include at least 1 nM of polypeptide. In other examples, the composition can include at least 5 μM, at least 50 μM, or at least 500 μM of the polypeptide. In yet another example, the composition can include between 1 μM and 1 mM of polypeptide.

The composition can further comprise reagents including one or more of a reducing agent, a buffer, a salt, and solvent. The macromolecule can be treated with a reducing agent such as tris(2-carboxyethyl)phosphine hydrochloride (TCEP·HCl, CAS No. 51805-45-9) to reduce any disulfide bonds that may have formed during storage or preparation of the macromolecule. Treatment with TCEP increases the number of alkyl thiol groups available for modification with a PEG group. The reducing agent can be for example, TCEP, tris(3-hydroxypropyl)phosphine (THPP), or thioglycolic acid.

The salt can include salts of ammonium, alkali metals, alkali earth metals, and transition metals. The salt can include biologically common anions such as chloride, nitrate, acetate, carbonate, citrate, EDTA, sulfate, and phosphate.

The buffer can be biological buffers typically used for proteins and peptides, including TRIS, HEPES, MOPS, MES, BES, MOPSO, ACES, TAPS, citrate, or bicine. Solvents for the buffer can include water, D20, acetonitrile, DMSO, methanol, or ethanol. Other additives can include guanidine, trifluoroacetic acid, or formic acid.

The composition can have a pH of between 0.5 and 14, between 2 and 12, between 4 and 10, or between 5.5 and 7.5. Suitably, the composition can have a pH in a range where the subject protein is stable.

The composition can further comprise a cysteine-aryl conjugated polypeptide. In an example, a residue comprising a free thiol group, such as a cysteine residue, of the polypeptide is conjugated to an aryl group having a PEG substituent. The cysteine-aryl conjugated polypeptide may be the product of the methods disclosed herein.

A method of preparing a cysteine-aryl conjugated polypeptide can be disclosed as follows. The method can include contacting the gold(III) aryl complex and the polypeptide. The gold(III) aryl complex and the polypeptide can be combined under conditions sufficient to prepare the cysteine-aryl conjugated polypeptide. The gold(III) aryl complex and the polypeptide can be combined in the molar ratio amounts according to the composition described above such that the gold(III) aryl complex and the polypeptide are contacted efficiently to produce the cysteine-aryl conjugated polypeptide. The gold(III) aryl complex and the polypeptide can be combined in the concentrations as described above for efficient contact to produce the cysteine-aryl conjugated polypeptide. The gold(III) aryl complex and the polypeptide can be combined with the reagents such as buffer, solvent, and salts according to the composition described above.

For example, the gold(III) aryl complex and the polypeptide can be contacted at a temperature less than 30° C. to produce the cysteine-aryl conjugated polypeptide. In another example the gold(III) aryl complex and the polypeptide can be contacted at a temperature less than 20° C., less than 10° C., or less than 5° C. to produce the cysteine-aryl conjugated polypeptide.

According to an aspect disclosed herein, the gold(III) aryl complex and the polypeptide are contacted for a time less than 5, 4, 3, 2, or 1 minutes to produce the cysteine-aryl conjugated polypeptide. Under these conditions the gold(III) aryl complex and the polypeptide produce the cysteine-aryl conjugated polypeptide. The conditions are sufficient to convert at least 90% of the polypeptide to the S-aryl conjugated polypeptide.

According to an aspect, the method further includes purifying the cysteine-aryl conjugated polypeptide. The cysteine-aryl conjugated polypeptide can be purified by size exclusion-fast protein liquid chromatography, dialysis, or spin filtration. Other methods for purifying the cysteine-aryl conjugated polypeptide may also be utilized.

When the gold(III) aryl complex and the polypeptide are contacted, the gold(III) aryl complex forms sulfur-carbon bonds rapidly and selectively between the aryl group and thiols via reductive elimination. The relative thiophilicity of Au(III), as well as the reluctance of the Au(I) byproduct to undergo oxidative addition provided the benefit of minimal background reactivity with the many reactive substrates present in a biological setting.

The gold(III) aryl complex utilized in the composition and methods described herein may be prepared by heating a composition including gold(I) precursor, a halide scavenger, and a pegylated aryl iodide. The gold(I) precursor can be for example (MeDalPhos)AuCl. The pegylated aryl iodide can have a polyethylene glycol group having an average molecular weight greater than 1 kDa attached to an aryl group. The halide scavenger can be a silver salt such as AgSbF₆, silver hexafluoroantimonate(V). The composition can include an excess such as a molar excess of both the gold (III) precursor and halide scavenger relative to the pegylated aryl iodide.

Treatment of a pegylated aryl iodide reagent with (Me-DalPhos)Au(I)Cl in the presence of AgSbF₆ results in oxidative addition across the terminal mPEG aryl-iodide bond thereby generating (mPEG)-Au(III)(Me-DalPhos). Acting as a halide scavenger in this chemical reaction, AgSbF₆ combines with (Me-DalPhos)Au(I)Cl to produce AgCl as a precipitated solid which can be readily separated from (mPEG)-Au(III)(Me-DalPhos) by filtration. Additional and alternative examples of halide scavengers include silver hexafluorophosphate, silver acetate, silver nitrate, silver tetrafluoroborate, and silver trifluoromethanesulfonate.

The synthesis of PEG-Au(III) polymer reagents is often plagued by long reaction times, harsh conditions, and low levels of conversion due to their large size. In the case of small molecules, excess PEG-aryl iodide is used to help achieve quantitative conversion to Au(III) reagent. In this disclosure, excess PEG-aryl iodide polymer cannot be used in the oxidative addition step, as it is challenging to purify away the excess polymer from the Au(III) polymer product. For this reason, excess (Me-DalPhos)Au(I)Cl and AgSbF₆ are used along with heat to achieve quantitative conversion. The purification strategy is designed to remove excess Au(I) and Ag from the resulting product rather than excess PEG-aryl iodide.

When conjugating polymers to proteins using the grafting to approach, it is helpful to avoid large excess of polymer reagent, as the purification of excess polymer from the conjugate is challenging due to the large size of each component. Additionally, the instability of proteins at room temperature is a common problem when considering reaction times and temperatures for these transformations. For this reason, the reductive elimination process to using minimal amounts of polymer reagent to achieve high conversion to these conjugates in minutes under mild conditions is particularly advantageous. The conditions disclosed herein result in near quantitative conversion to conjugate using near equimolar amounts of reagent in just one minute at both room temperature and 4° C. using this technique, making this a desirable approach for the synthesis of a broad range of protein polymer conjugates.

Unless otherwise specified or indicated by context, the terms “a”, “an”, and “the” mean “one or more.” For example, “a molecule” should be interpreted to mean “one or more molecules.” As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein. Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Examples

### Materials and Methods

Unless otherwise noted, all materials were of analytical grade and purchased and used as received from Fisher Scientific, Acros Organics, Oakwood Chemicals or Sigma Aldrich. The silver hexafluoroantimonate (AgSbF₆) was stored in the glovebox under an atmosphere of N₂ and removed prior to use. Milli-Q water was used for all experiments. Fisher Water Optima™ LC-MS Grade and Fisher Acetonitrile Optima™ LC-MS Grade were used exclusively for LC-MS mobile phase solvents.

NMR spectra were recorded on AV 400 and DRX 500 Bruker spectrometers at 400 or 500 MHz (¹H) and 121 MHz (³¹P{¹H}). Spectra are reported in 6 (parts per million) relative to residual proteo-solvent signals for ¹H and H₃PO₄ (δ 0.00 ppm) for ³¹P{¹H}. Deuterated solvents (Cambridge Isotope Laboratories) were used for all NMR experiments. ¹H NMR spectra for all 10 kDa mPEG polymers were acquired with a relaxation delay of 10 seconds. For all smaller mPEG polymers, spectra were acquired with a relaxation delay of 4 seconds.

DARPin and DARPin mPEG conjugates were purified by FPLC on a Bio-Rad BioLogic DuoFlow chromatography system. All purifications were carried out at 4° C. All buffers were freshly prepared and filtered over a Thermo Scientific Nalgene 565-0020 Filter Unit, 0.2 um PES prior to use. Size exclusion chromatography (SEC) purifications were performed using a Superdex 75 Increase 10/300 GL column. All protein purifications were monitored at wavelengths of 254 nm and 280 nm. A standard isocratic method was used for all sampled (20 mM Tris, 150 mM NaCl buffer, pH 7.5 over 50 minutes).

Protein concentration measurements were determined on a NanoDrop 2000 UV-Vis spectrophotometer at 280 nm. Extinction coefficients were calculated by ProtoParam on ExPASy based on the amino acid sequence of the protein (DARPin=15,470 M⁻¹ cm⁻¹).

Sodium dodecyl sulfate-polyacrylamide gel electrophoresis (SDS-PAGE) was carried out in a Mini-PROTEAN Tetra Cell system (Bio-Rad) connected to a PowerPac HC (BioRad) power supply using Bio-Rad Any kD™ Mini-PROTEAN® TGX™ Precast Gels at 195 V and 3 A for 30 minutes in a running buffer (25 mM Tris, 192 mM Glycine, 0.1% (w/v) SDS, pH 8.3). Precision Plus Protein™ Dual Xtra Prestained Protein Standards (2 μL) were used as protein ladder in all SDS-PAGE analysis. Laemmli 2× Concentrate (Sigma) containing 4% SDS, 20% glycerol, 0.004% bromophenol blue and 0.125 M Tris HCl at a pH of approximately 6.8 was used to load all protein and conjugate samples. Protein bands were visualized by staining the gels in an aqueous solution (0.1% Coomassie Brilliant Blue R 250, 45% MeOH, 9% acetic acid) and microwaving for 30 seconds followed by agitation for 15 minutes. Destaining was carried out by submerging the gels in an aqueous destaining solution (10% MeOH, 14% acetic acid), microwaving for 30 seconds, and agitating for several hours until the background of the gel became fully destained. ImageJ was used to calculate conversion by optical densitometry.

LC-MS analysis was carried out using an Agilent 6530 ESI-Q-TOF. DARPin analyses were carried out using an Agilent ZORBAX 300SB C3 column (3.5 μm, 3.0×150 mm). Liquid chromatography method used for proteins: Column temperature: 40° C. Flow rate: 0.5 mL/min. Gradient: 90% water (0.1% formic acid (FA)) for 2 minutes; 90%-9% water (0.1% FA) 2-11 minutes; 5% water (0.1% FA) from 11-12 min. Sample preparation: For 70 μM reactions, 10 μL was added to 90 μL of 50:50 MeCN:H₂O with 0.1% formic acid. For 7 μM reactions, 50 μL was added to 50 μL of 50:50 MeCN:H₂O with 0.1% formic acid.

Circular dichroism was performed on a Chirascan V-100 equipped with a Peltier at a temperature of 25° C., unless otherwise noted.

MALDI-TOF spectra were collected using an Applied Biosystems Voyager-DEtm STR instrument with instrument settings optimized for positive ion, linear mode, 20 kV accelerating voltage, 94 grid voltage, 100 nsec delay time and typically 500 laser shots per spectrum with external calibration using cytochrome c.

Gold ICP-OES analyses were conducted using an Agilent 5110 ICP-OES (inductively coupled plasma-optical emission spectrometer). A Sigma-Aldrich 1000 ppm (Lot value: 999 ppm±2 ppm, 5% w/w HCl) Gold Standard for ICP was used as a stock solution to create standards of concentrations 50 ppb, 300 ppb, and 600 ppb. Solutions were prepared using a 50.00 mL volumetric flask and Eppendorf pipettes for aliquoting of the Au stock solution. A calibration curve was generated for each standard by integrating the signal corresponding to the characteristic Au emission (242.79 nm).

Synthesis of mPEG-Au(III) Reagents

Commercial mPEG reagents of 2, 5 and 10 kDa molecular weights were first subjected to tosylation conditions, followed by reaction with base and iodophenol to generate mPEG-aryl iodide reagents of each size in 74%, 82% and 78% isolated yield, respectively.

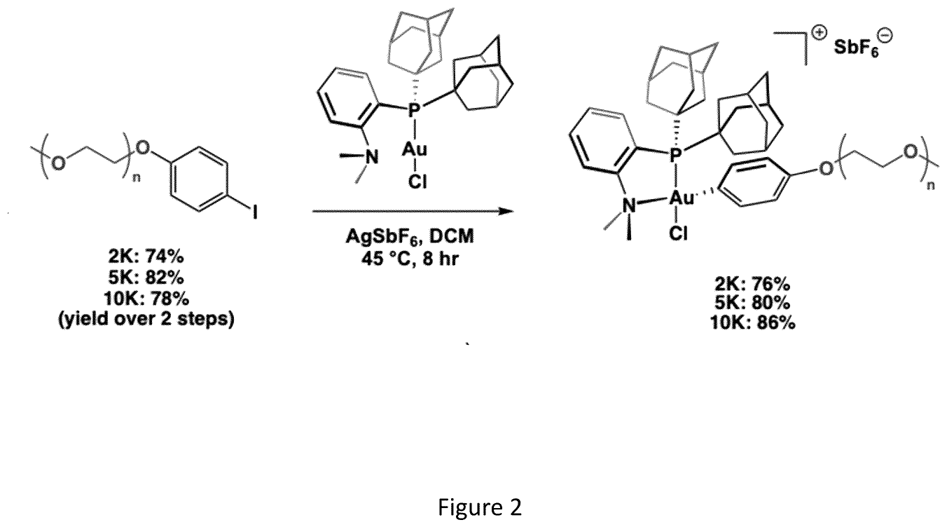

Treatment of mPEG-aryl iodide reagents with (Me-DalPhos)Au(I)Cl in the presence of the halide scavenger silver hexafluoroantimonate(V) (AgSbF₆) results in oxidative addition across the terminal mPEG aryl-iodide bonds thereby generating 2, 5 and 10 kDa mPEG-Au(III) reagents in quantitative conversions and high isolated yields (FIG. 2a). Excess aryl iodide has been utilized to promote the traditionally slow oxidative addition step thereby allowing quantitative conversion of the Au(III) complexes. However, because it is challenging to purify away the excess aryl iodide polymer from the Au(III) polymer product, further reaction optimization was needed. Extending the reaction time and applying heat enabled the quantitative conversion of mPEG-Au(III) reagents using sub-stoichiometric amounts of the aryl iodide polymer starting material.

### Synthesis of (Me-DalPhos)Au(I)Cl^{1, 2}

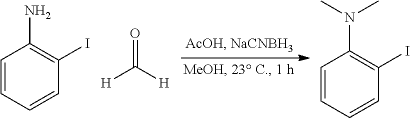

A one neck 1 L round bottom flask was charged with a stir bar, then 2-iodoaniline (10 g, 1 Eq, 45.6 mmol) was dissolved in 400 mL of methanol. Formaldehyde (34 mL, 10 Eq, 456 mmol, 37% weight in water) and acetic acid (13.1 mL, 5 equiv, 228 mmol) were added to the flask and left to stir for ten minutes at 23° C. Sodium cyanoborohydride (11.5 g, 4 equiv, 182 mmol) was added in portions over a 15 minute period, then this was left to stir for one hour at 23° C. The reaction was concentrated under vacuum and carefully pH adjusted to pH 8.0 using 1 M NaHCO₃. The product was transferred to a separatory with ethyl acetate (300 mL), then the organic layer was washed with NaHCO₃ (2×75 mL) and brine (100 mL). The organic layer was collected, dried with anhydrous magnesium sulfate, filtered, and concentrated under vacuum. The resulting product was further purified through a vacuum distillation at 85° C. to afford a clear liquid (9.445 g, 84% yield). 1H NMR (400 MHz, CDCl3): δ 7.84 (dd, J=7.8, 1.5 Hz, 1H), 7.31 (m, 1H), 7.10 (dd, J=8.0, 1.5 Hz, 1H), 6.77 (m, 1H), 2.77 (s, 6H). 13C NMR (101 MHz, CDCl3): δ 155.10, 140.33, 129.19, 125.12, 120.61, 97.27, 45.12. Spectra match those of literature values.3

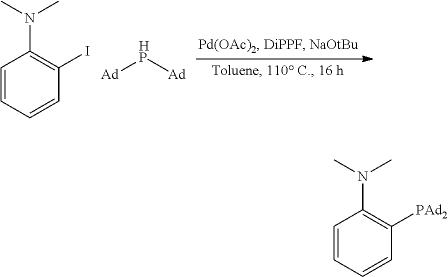

Inside of a nitrogen atmosphere in a glovebox, Pd(OAc)₂ (16.7 mg, 0.025 Eq, 74.4 μmol) and DiPPF (37.3 mg, 0.030 equiv, 89.3 μmol) were added as solids to a dram vial charged with a stir bar, then the solids were dissolved in 400 μL of anhydrous toluene and left to stir at 23° C. for 15 minutes. Separately, di-1-adamantylphosphine (900 mg, 1 equiv, 2.98 mmol) and NaOtBu (429 mg, 1.5 Eq, 4.46 mmol) were added as solids to a dram vial charged with a stir bar and subsequently dissolved in 8 mL of anhydrous toluene where this was left to stir for 15 minutes. 2-iodo-N,N-dimethylaniline (757 mg, 103 equiv 3.07 mmol) was weighed out into a scintillation vial charged with a stir bar and diluted with 2 mL of anhydrous toluene. The Pd(OAc)₂/DiPPF solution was transferred to the scintillation vial, then the HPAd₂/NaOtBu solution was transferred to the scintillation vial. Both dram vials were washed with 500 μL (1 mL total) of anhydrous toluene. The scintillation vial was sealed with electrical taped and removed from the glovebox where it was refluxed in a closed vial at 110° C. for 16 hours. The reaction was cooled to 23° C., and the reaction was concentrated under vacuum. The solid was redissolved in chloroform and filtered through a plug of celite, then the product was concentrated under vacuum. The product was suspended in cold hexanes and transferred to a fritted funnel where the product was rinsed with cold diethyl ether (10 mL), cold acetonitrile (10 mL), and cold diethyl ether (10 mL). The solid was then dried under vacuum to afford the product as a white solid (893 mg, 71% yield). ¹H NMR (400 MHz, CDCl₃) δ 7.71 (dt, J=7.6, 1.5 Hz, 1H), 7.36-7.29 (m, 1H), 7.20 (ddd, J=8.1, 4.6, 1.2 Hz, 1H), 7.05 (td, J=7.4, 1.3 Hz, 1H), 2.71 (s, 6H), 2.03-1.85 (m, 18H), 1.67 (s, 12H). ³¹P{¹H} NMR (121 MHz, CD₂Cl₂): δ 19.99 ppm. Spectra match those of literature values.

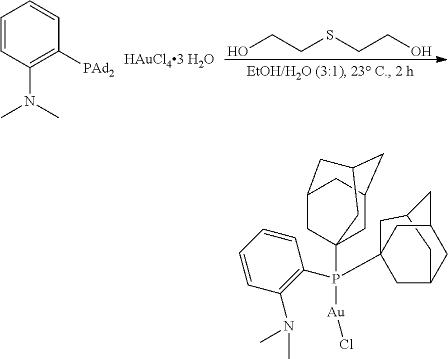

A scintillation vial was charged with a stir bar and HuC₄·3H₂O (32 mg, 1 Eq, 819 μmol) was added as a solid to the flask, then it was dissolved in 2 mL of DI water. Separately, Me-DalPhos (345 mg, 1 equiv, 819 μmol) was suspended in 3 mL of ethanol in a dram vial, then this was added to the scintillation vial. The dram vial was washed with ethanol (3×1 mL) and transferred to the scintillation vial. This was stirred at 23° C. for two hours. The contents of the reaction were then transferred onto a fritted funnel where the white solid was washed with methanol (15 mL). The white solid was dissolved in DCM and filtered through a plug of celite to remove any nanoparticles that may have formed. The eluent was then concentrated under vacuum to afford the product as a white solid (482 mg, 90% yield). ¹H NMR (400 MHz, CDCl₃) δ 7.74 (ddd, J=8.0, 6.6, 1.5 Hz, 1H), 7.59-7.50 (m, 2H), 7.29 (ddd, J=8.3, 4.8, 1.8 Hz, 1H), 2.60 (s, 6H), 2.21 (ddd, J=11.7, 5.4, 2.9 Hz, 6H), 2.09 (ddt, J=12.1, 6.0, 2.8 Hz, 6H), 1.98 (d, J=2.9 Hz, 6H), 1.68 (s, 12H). ³¹P{¹H} NMR (121 MHz, CD₂Cl₂): δ 56.54 ppm. Spectra match those of literature values.²

Synthesis of Au(I) mPEG Reagents

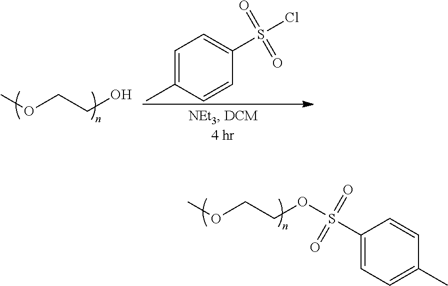

General Procedure A:⁴ A round bottom flask equipped with a Teflon coated magnetic stir bar was charged with a solution of poly(ethylene glycol) methyl ether in anhydrous dichloromethane. Triethylamine was added followed by 4-methylbenzenesulfonyl chloride. The reaction was stirred for 4 hr at 25° C. The solution was then concentrated under reduced pressure and precipitated from cold diethyl ether. The precipitate was collected, redissolved in DCM, and precipitated again from cold ether. This process was repeated once more to afford a mixture of triethyl ammonium HCl salts and the desired product as a white solid which was used without further purification. All spectra match those of literature values.

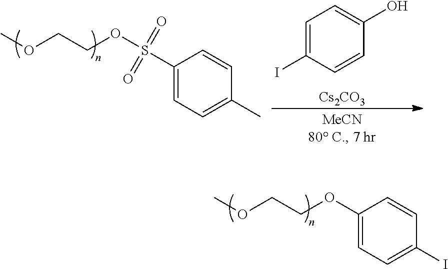

General Procedure B: OTs-mPEG and 4-iodophenol were added to an oven dried round bottom flask with an appropriately sized stir bar and were dissolved in anhydrous MeCN. To this solution was added Cs₂CO₃ while stirring. The reaction mixture was heated to 80° C. and was stirred for 7 hours, at which point the solvent was removed under reduced pressure to afford light pink solids. The crude mixture was dissolved in DCM and filtered to remove excess Cs₂CO₃. The DCM was removed under vacuum and the remaining light pink solids were dissolved in a small amount of water, and the basic solution was neutralized to pH 7-8 with 1 M NH₄OAc buffer. The aqueous solution was removed on the lyophilizer to provide white solids. These solids were resuspended in DCM and filtered to remove undissolved salts. The DCM solution was concentrated under reduced pressure and precipitated from cold diethyl ether two times (to remove excess iodophenol), then once at in −78° C. ethanol (to remove tosylic acid byproduct), then again once in cold diethyl ether. All spectra match those of literature values.

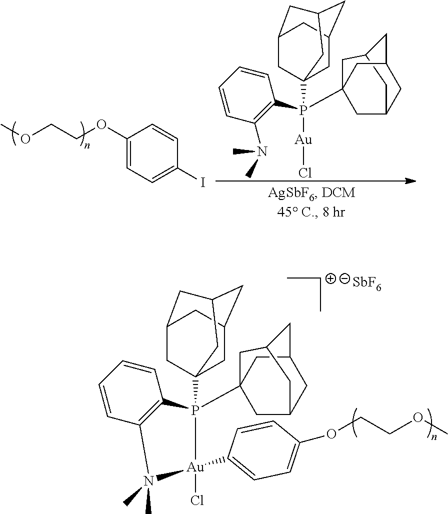

General Procedure C: AgSbF₆ was dissolved in DCM (1 mL) under protection from light, and the solution was cooled to −20° C. A DCM solution (1 mL) containing the mPEG-aryl-I and (Me-DalPhos)AuCl reagents was prepared and also cooled to −20° C. While both solutions were cold, the colorless mPEG-aryl-I and (Me-DalPhos)AuCl solution was added in one portion to the solution of AgSbF₆, resulting in visible precipitation of pale-yellow solids. The reaction was stirred for 8 hours at 45° C. Then, the reaction mixture was filtered through a pad of Celite to remove Ag salts. The solution was concentrated under reduced pressure and precipitated from cold (−78° C.) tetrahydrofuran twice to afford the product as a pale-yellow solid (the larger polymers have a less bright yellow color). If full end-group conversion was not reached after 8 hours, the purified mixture of Au(III) mPEG product and mPEG aryl iodide was resubjected to the same reaction conditions until fully converted.

Tosylation of mPEG-OH Reagents

2 kDa mPEG-OTs: Following General Procedure A, 2 kDa mPEG (1.0 g, 0.50 mmol, 1 equiv) and triethylamine (0.35 mL, 2.5 mmol, 5 equiv) in 8 mL DCM and 4-methylbenzenesulfonyl chloride (476 mg, 2.5 mmol, 5 equiv) in 2 mL DCM were used. The product was isolated as a white solid. 1H NMR (400 MHz, CDCl₃) δ 7.75-7.71 (m, 2H), 7.28 (m, 2H), 4.11-4.07 (m, 2H), 3.58 (m, 188H), 3.31 (s, 3H), 2.39 (s, 3H).

5 kDa mPEG-OTs: Following General Procedure A, 5 kDa mPEG (1.0 g, 0.20 mmol, 1 equiv) and TEA (0.140 mL, 1 mmol, 5 equiv) in 2 mL DCM and 4-methylbenzenesulfonyl chloride (191 mg, 1 mmol, 5 equiv) in 2 mL DCM were used. The product was isolated as a white solid. ¹H NMR (500 MHz, CDCl₃) δ 7.79-7.75 (m, 2H), 7.32 (m, 2H), 4.15-4.11 (m, 2H), 3.62 (m, 483H), 3.36 (s, 3H), 2.43 (s, 3H).

10 kDa mPEG-OTs: Following General Procedure A, 10 kDa mPEG (500 mg, 50 umol, 1 equiv) and TEA (35 μL, 250 umol, 5 equiv) in 3 mL DCM and 4-methylbenzenesulfonyl chloride (42.5 mg, 250 umol, 5 equiv) in 2 mL DCM were used. The product was isolated as a white solid. ¹H NMR (400 MHz, CDCl₃) δ 7.75-7.70 (m, 2H), 7.31-7.26 (m, 2H), 4.11-4.06 (m, 2H), 3.57 (m, 1010H), 3.31 (s, 3H), 2.38 (s, 3H).

Arylation of mPEG-OTs Reagents

2 kDa mPEG-ArI: Following General Procedure B, 2 kDa mPEG-OTs (500 mg, 0.238 mmol, 1 equiv) and 4-iodophenol (157 mg, 0.714 mmol, 5 equiv) were dissolved in 5 mL MeCN. Cs₂CO₃ (233 mg, 0.714 mmol, 5 equiv) was added to the flask with a rounded bottom while stirring. The product was isolated as a white solid in 74% yield. ¹H NMR (500 MHz, CDCl₃) δ 7.50-7.45 (m, 2H), 6.67-6.62 (m, 2H), 4.06-4.00 (m, 2H), 3.78 (m, 2H), 3.59 (m, 185H), 3.32 (s, 3H).

5 kDa mPEG-ArI: Following General Procedure B, 5 kDa mPEG-OTs (500 mg, 0.098 mmol, 1 equiv) and 4-iodophenol (108 mg, 0.49 mmol, 5 equiv) were dissolved in 2 mL MeCN. Cs₂CO₃ (160 mg, 0.49 mmol, 5 equiv) was added to the flask with a rounded bottom while stirring. The product was isolated as a white solid in 82% yield. ¹H NMR (400 MHz, CDCl₃) δ 7.55-7.48 (m, 2H), 6.70-6.64 (m, 2H), 4.08-4.03 (m, 2H), 3.61 (m, 471H), 3.35 (s, 3H).

10 kDa mPEG-ArI: Following General Procedure B, 10 kDa mPEG-OTs (1 g, 0.099 mmol, 1 equiv) and 4-iodophenol (109 mg, 0.50 mmol, 5 equiv) were dissolved in 2 mL MeCN. Cs₂CO₃ (161 mg, 0.50 mmol, 5 equiv) was added to the flask with a rounded bottom while stirring. The product was isolated as a white solid in 78% yield. ¹H NMR (400 MHz, CDCl₃) δ 7.53-7.46 (m, 2H), 6.68-6.63 (m, 2H), 4.07-4.03 (m, 2H), 3.60 (m, 839H), 3.34 (s, 3H).

Oxidative Addition of mPEG-ArI Reagents

2 kDa mPEG (Me-DalPhos)Au(III): Following General Procedure C, (Me-DalPhos)AuCl (30 mg, 0.046 mmol, 2 equiv), AgSbF₆ (13 mg, 0.039 mmol, 1.7 equiv) and 2 kDa mPEG-aryl-I (50 mg, 0.023 mmol, 1 equiv) were used. The product was isolated as a yellow solid in 76% yield. ¹H NMR (500 MHz, CDCl₃) δ 8.03-7.89 (m, 3H), 7.78 (t, J=7.2 Hz, 1H), 7.33-7.28 (m, 2H), 6.95-6.87 (m, 2H), 4.17-4.11 (m, 2H), 3.90-3.86 (m, 2H), 3.48-3.75 (m, 188H), 3.50 (s, 6H), 3.38 (s, 3H), 2.26 (m, 6H), 2.11 (m, 12H), 1.75 (m, 12H). ³¹P{¹H} NMR (121 MHz, CD₂Cl₂): δ 74.64 ppm.

5 kDa mPEG (Me-DalPhos)Au(III): Following General Procedure C, (Me-DalPhos)AuCl (30 mg, 0.046 mmol, 2 equiv), AgSbF₆ (13 mg, 0.039 mmol, 1.7 equiv) and 5 kDa mPEG-aryl-I (120 mg, 0.023 mmol, 1 equiv) were used. The product was isolated as a yellow solid in 80% yield. ¹H NMR (500 MHz, CDCl₃) δ 8.03-7.89 (m, 3H), 7.76 (t, J=8.1 Hz, 1H), 7.29 (d, J=8.8 Hz, 2H), 6.90 (d, J=8.7 Hz, 2H), 4.13 (t, J=4.7 Hz, 2H), 3.48-3.76 (m, 562H), 3.49 (s, 6H), 3.36 (s, 3H), 2.24 (m, 6H), 2.10 (m, 12H), 1.78-1.70 (m, 12H). ³¹P{¹H} NMR (121 MHz, CD₂Cl₂): δ 74.75 ppm.

10 kDa mPEG (Me-DalPhos)Au(III): Following General Procedure C, (Me-DalPhos)AuCl (15 mg, 0.024 mmol, 2 equiv), AgSbF₆ (6.8 mg, 0.020 mmol, 1.7 equiv) and 5 kDa mPEG-aryl-I (120 mg, 0.012 mmol, 1 equiv) were used. The product was isolated as a yellow solid in 86% yield. ¹H NMR (400 MHz, CDCl₃) δ 8.08 (dd, J=8.5, 4.1 Hz, 1H), 8.00 (t, J=7.7 Hz, 1H), 7.92 (t, J=7.7 Hz, 1H), 7.78 (t, J=7.6 Hz, 1H), 7.29 (d, J=8.9 Hz, 2H), 6.89 (d, J=8.6 Hz, 2H), 4.12 (t, J=4.7 Hz, 2H), 3.47-3.81 (m, 934H), 3.36 (s, 3H), 2.25 (m, 6H), 2.10 (m, 12H), 1.79-1.71 (m, 12H). ³¹P{¹H} NMR (121 MHz, CD₂Cl₂): δ 74.70 ppm.

### DARPin Expression

DARPin-Cys protein expression and purification was adapted from literature procedures.⁵ DARPin-Cys Sequence (Calculated Mass: 15996.84 Da).

(SEQ ID NO: 1)
MGSDKIHHHHHHENLYFQGGCGGSDLGKKLLEAARAGQDDEVRILMANG
ADVNAYDDNGVTPLHLAAFLGHLEIVEVLLKYGADVNAADSWGTTPLHL
AATWGHLEIVEVLLKHGADVNAQDKFGKTAFDISIDNGNEDLAEILQKL
N

The plasmid was designed to have an N-terminal His₆ tag followed by a TEV protease cleavage site which was left on for our purposes (model bioconjugation protein). The plasmid purchased from Twist Bioscience is a pET29b(+) vector with kanamycin resistance and the DARPin-Cys gene was cloned in via NdeI and XhoI restriction sites.

Prior to expression, the plasmid was transformed into BL21-Gold cells (Agilent) using the standard manufacturer's procedure. Overnight cultures were grown and from these, two glycerol stocks were made and stored in the −80° C. freezer. To each of two 2 L flasks each containing 750 mL of previously autoclaved LB Broth (Miller) with kanamycin (50 μg/mL) was added 5 mL of a saturated overnight culture inoculated from one of the aforementioned glycerol stocks. The culture was grown at 37° C. with 250 rpm shaking for about 4 hours before the OD600 reached ˜0.4 and the culture was induced with 1 mM IPTG. The temperature was lowered and the culture was continued to shake at 30° C. at 250 rpm for approximately 17 hours. The final OD600 was observed to be 1.39. Uninduced and induced cell pellets from −3 mL of culture each were normalized to 1 OD600 unit/mL and analyzed by SDS-PAGE and Coomassie Blue staining. The cultures were harvested by centrifugation at 6000 rpm for 30 min to yield a cell pellet.

The pellet was resuspended in lysis buffer containing Tris and 150 mM NaCl (pH 7.5), 15 mg lysozyme and 0.5 tablet of protease inhibitor cocktail. The resulting suspension was homogenized (Avestin Emulsiflex C-3) and centrifuged at 17,000 rpm for 30 min to remove cell debris. The supernatant was loaded onto a 5 mL gravity Ni-NTA column (Qiagen) and washed with 30 mL (10 mL×3) 5 mM imidazole, 20 mL (10 mL×2) 20 mM imidazole and eluted with 25 mL (5 mL×5) 200 mM imidazole all in 20 mM Tris 150 mM NaCl pH 8.5 buffer. SDS-PAGE was run on all fractions and under reducing conditions with Coomassie Blue staining. Pure fractions were combined and solvent exchanged into storage buffer (20 mM Tris, 150 mM NaCl, pH 7.5) and concentrated to −15 mL using a using Amicon 3K Ultra-15 Centrifugal Filter (Millipore).

The purified protein was analyzed by LC-MS* and SDS-PAGE confirming sample purity and molecular weight. Concentration was determined by A280 (Extinction coefficient=15470 M-1 cm-1) and confirmed by Ellman's (5,5′-dithio-bis-(2-nitrobenzoic acid)) Assay after treatment with and subsequent removal of two equivalents TCEP·HCl (Tris (2-carboxyethyl) phosphine hydrochloride) (Amicon 3K Ultra-0.5 mL Centrifugal Filters). The protein sample was diluted with storage buffer to 300 μM and aliquots were flash frozen and stored in a −80° C. freezer. The observed mass by LC-MS of purified protein is 15,866 Da corresponding to the calculated mass of the sequence without the initial methionine which was likely cleaved during expression.⁶

### Conjugation Condition Screening

Standard conjugation conditions: (70 μM DARPin, pH 7.5, 4 equiv. TCEP, 1.3 equiv. Au(III), 1 minute, 25° C.) To 50 μL of a solution of 76 μM DARPin in 20 mM Tris, 150 mM NaCl buffer, pH 7.5, was added 5 μL of a 3 mM stock solution of TCEP-HCl (4 equivalents) in MilliQ H₂O. This solution was allowed to sit at 25° C. for one hour to reduce any disulfide bonds formed during storage. Then, 5 μL of a 1 mM stock solution of Au(III) mPEG reagent (1.3 equivalents) in MilliQ H₂O was added, gently flicked to mix and allowed to react for one minute. After one minute, the reaction was stopped by dilution in appropriate media for analysis (see below) or subjection to FPLC purification.

Large scale conjugation conditions: To 200 μL of a solution of 7.6 μM DARPin in 20 mM Tris, 150 mM NaCl buffer, pH 7.5, was added 20 μL of a 3 mM stock solution of TCEP·HCl (4 equivalents) in MilliQ H₂O. This was allowed to sit at 25° C. for one hour to reduce any disulfide bonds formed during storage. Then, 20 μL of a 1 mM stock solution of Au(III) mPEG reagent (1.3 equivalents) in MilliQ H₂O was added, gently flicked to mix and allowed to react for one minute.

General SDS-PAGE procedure: After one minute, 1 μL of the reaction mixture was diluted into 19 μL Laemmli loading buffer in preparation for SDS-PAGE analysis. Samples were loaded onto SDS-PAGE gel and run at 195 V for 30 min and Coomassie stained.

### Au(III) PEGylation Au(III) Equivalent Screen

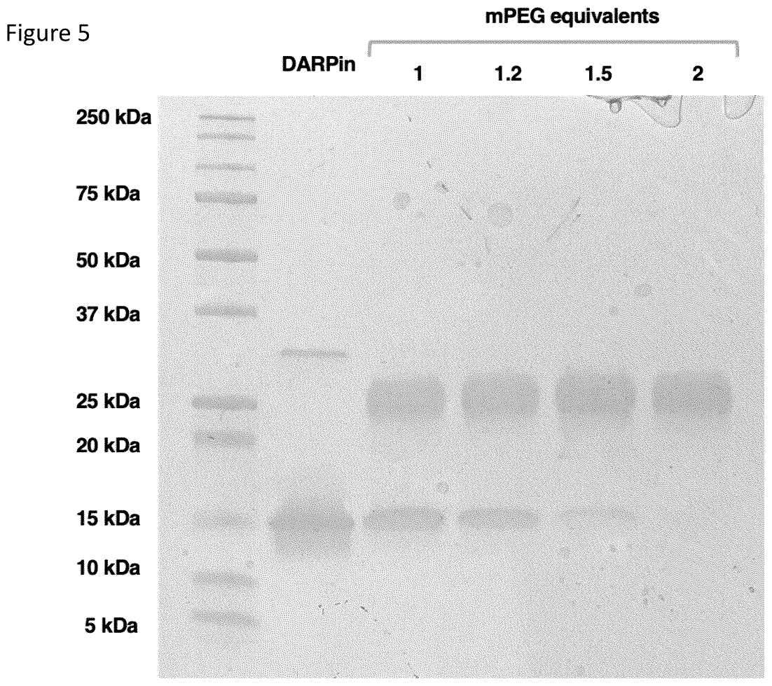

Au(III) equivalent conjugation screen: (70 μM DARPin, pH 7.5, 4 equiv. TCEP, X equiv. Au(III), 1 minute, 25° C.) To 50 μL of a solution of 76 μM DARPin in 20 mM Tris, 150 mM NaCl buffer, pH 7.5, was added 5 μL of a 3 mM stock solution of TCEP·HCl (4 equivalents) in MilliQ H₂O. This was allowed to sit at 25° C. for one hour to reduce any disulfide bonds formed during storage. Then, 5 μL of a 0.76, 0.9, 1.2 or 1.5 mM stock solution of Au(III) mPEG (1, 1.2, 1.5 or 2 equivalents, respectively) reagent was added, gently flicked to mix and allowed to react for one 25 minute. FIG. 5 shows an SDS-PAGE of crude 5 kDa Au(III) PEG-DARPin conjugates synthesized as above with 1, 1.2, 1.5, and 2 equivalents of 5 kDa mPEG Au(III) reagents.

### Au(III) PEGylation at Acidic pH

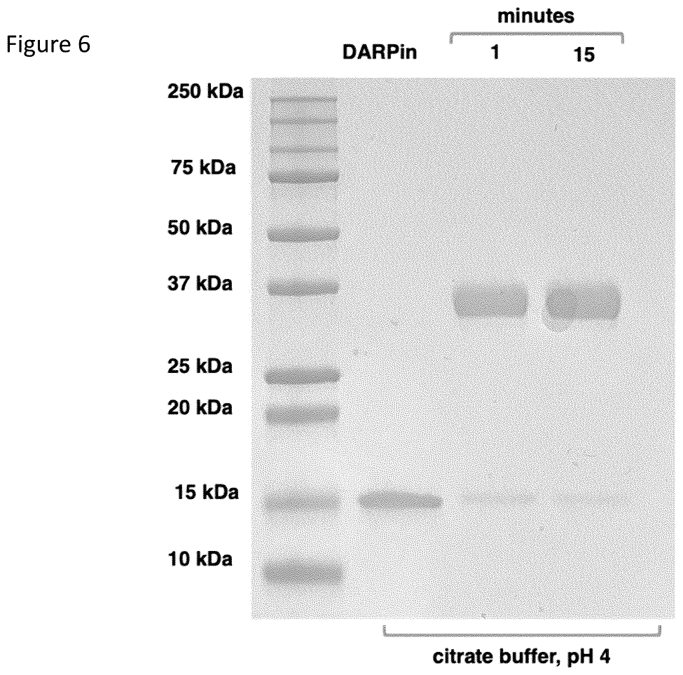

Acidic conjugation conditions: (70 μM DARPin, pH 5.5, 4 equiv. TCEP, 1.3 equiv. Au(III), 1 minute, 25° C.) To 50 μL of a solution of 76 μM DARPin in 100 mM citrate buffer, pH 5.5, was added 5 μL of a 3 mM stock solution of TCEP-HCl (4 equivalents) in MilliQ H₂O. This was allowed to sit at 25° C. for one hour to reduce any disulfide bonds formed during storage. Then, 5 μL of a 1 mM stock solution of Au(III) mPEG reagent (1.3 equivalents) in MilliQ H₂O was added, gently flicked to mix and allowed to react for one minute. FIG. 6 shows SDS-PAGE of crude 10 kDa Au(III) PEG-DARPin conjugates synthesized as above after one and 15 minutes at 25° C.

### Au(III) PEGylation Concentration and Temperature Screen

Dilute conjugation conditions: (7 μM DARPin, pH 7.5 4 equiv. TCEP, 1.3 equiv. Au(III), 1 minute, 25° C.) To 50 μL of a solution of 76 μM DARPin in 20 mM Tris, 150 mM NaCl buffer, pH 7.5, was added 5 μL of a 3 mM stock solution of TCEP-HCl (4 equivalents) in MilliQ H₂O. This was allowed to sit at 25° C. for one hour to reduce any disulfide bonds formed during storage. Then, 5 μL of the 76 μM reduced DARPin stock was added to 45 μL of 20 mM Tris, 150 mM NaCl buffer, pH 7.5 to make a 7.6 μM stock of reduced DARPin. Then, 5 μL of a 0.1 mM stock solution of Au(III) mPEG reagent (1.3 equivalents) in MilliQ H₂O was added, gently flicked to mix and allowed to react for one minute.

4° C. conjugation conditions: Reactions performed at 4° C. were carried out identically to the standard and dilute reaction conditions, with the exception of temperature. DARPin was first reduced with TCEP at room temperature for 1 hour and subsequently moved to a 4° C. refrigerator and allowed to cool for 30 minutes. The Au(III) reagents were then added and the aliquots for analysis were removed while inside the refrigerator.

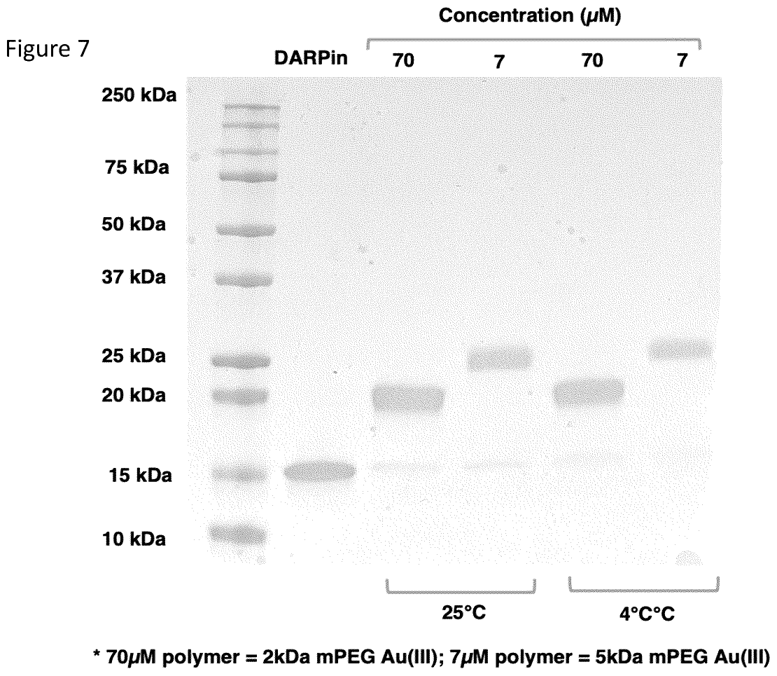

SDS-PAGE procedure: After one minute, 1 μL of the 70 μM solution was diluted into 19 μL Laemmli loading buffer or 10 μL of the 7 μM solution was diluted into 10 μL Laemmli loading buffer in preparation for SDS-PAGE analysis. Samples were loaded onto SDS-PAGE gel and run at 195 V for 30 min and Coomassie stained. FIG. 7 shows SDS-PAGE of crude 5 and 10 kDa Au(III) PEG DARPin conjugates synthesized in as above with 70 μM (5 kDa) and 7 μM (10 kDa) and at both 25° C. and 4° C.

The results are summarized in Table 1.

TABLE 1
Screening conditions for the reaction of DARPin with
mPEG-Au(III) reagents with percent conversion for 1.3
equivalents of Au(III) after 1 min reaction time.

| | polymer | | | | | |
|---|---|---|---|---|---|---|
| | MW | | | [protein] | temperature | % |
| entry | (kDa) | buffer | pH | μM | (° C.) | conversion |
| 1 | 2 | Tris | 7.5 | 70 | 25 | 90 |
| 2 | 5 | Tris | 7.5 | 70 | 25 | 92 |
| 3 | 10 | Tris | 7.5 | 70 | 25 | 91 |
| 4 | 5 | Tris | 7.5 | 7 | 25 | 93 |
| 5 | 2 | Tris | 7.5 | 70 | 4 | 94 |
| 6 | 5 | Tris | 7.5 | 7 | 4 | 92 |
| 7 | 10 | Citrate | 5.5 | 70 | 25 | 92 |

* % conversion calculated by optical densitometry of SDS-PAGE gels (SI)

Au(III) PEGylation vs. Maleimide Competition

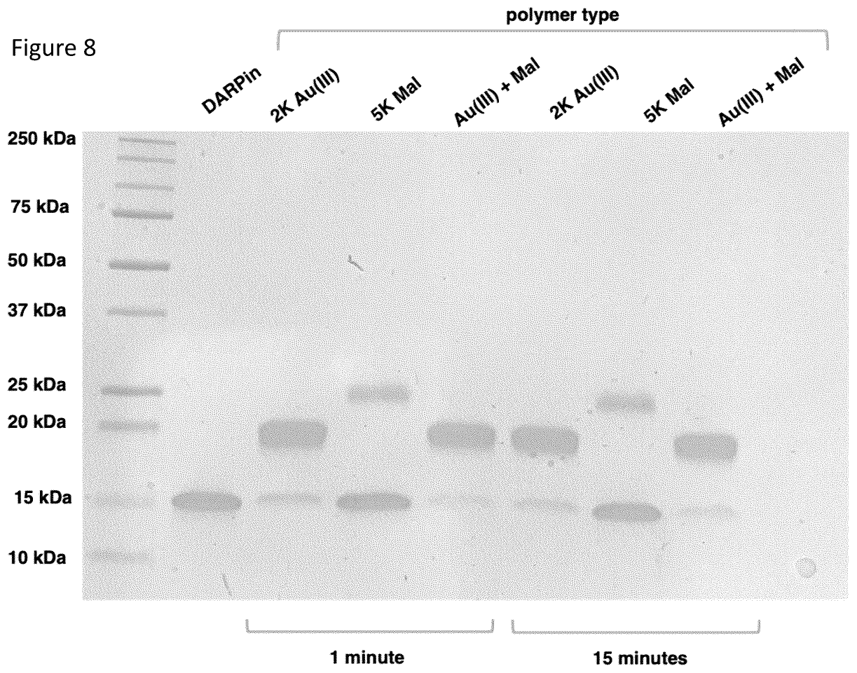

Maleimide competition: (70 μM DARPin, pH 7.5, 4 equiv. TCEP, 1.3 equiv. Au(III)/Mal, 1 minute, 25° C.) To 50 μL of a solution of 76 μM DARPin in 20 mM Tris, 150 mM NaCl buffer, pH 7.5, was added 5 μL of a 3 mM stock solution of TCEP·HCl in MilliQ H₂O (4 equivalents). This was allowed to sit at 25° C. for one hour to reduce any disulfide bonds formed during storage. Then, 5 μL of a 1 mM stock solution of either 2 kDa Au(III) mPEG or 5 kDa maleimide mPEG (1.3 equivalents) reagent was added, gently flicked to mix and allowed to react for one minute. In another reaction tube, 5 μL of both 2 kDa Au(III) mPEG and 5 kDa maleimide mPEG (1.3 equivalents) reagent were added, gently flicked to mix and allowed to react for one minute. SDS-PAGE: After one minute, 1 μL of the 70 μM solution was diluted into 19 μL into Laemmli loading buffer and run on SDS-PAGE. Samples were loaded onto SDS-PAGE gel and run at 195V for 30 min under and Coomasie stained. FIG. 8 shows an SDS-PAGE of crude 2 kDa Au(III) PEG-DARPin and 5 kDa maleimide PEG-DARPin conjugates synthesized as above with 70 μM solution after one and 15 minutes at 25° C.

Characterization of mPEG DARPin Conjugate
LC-MS Analysis of Crude mPEG-DARPin Conjugates

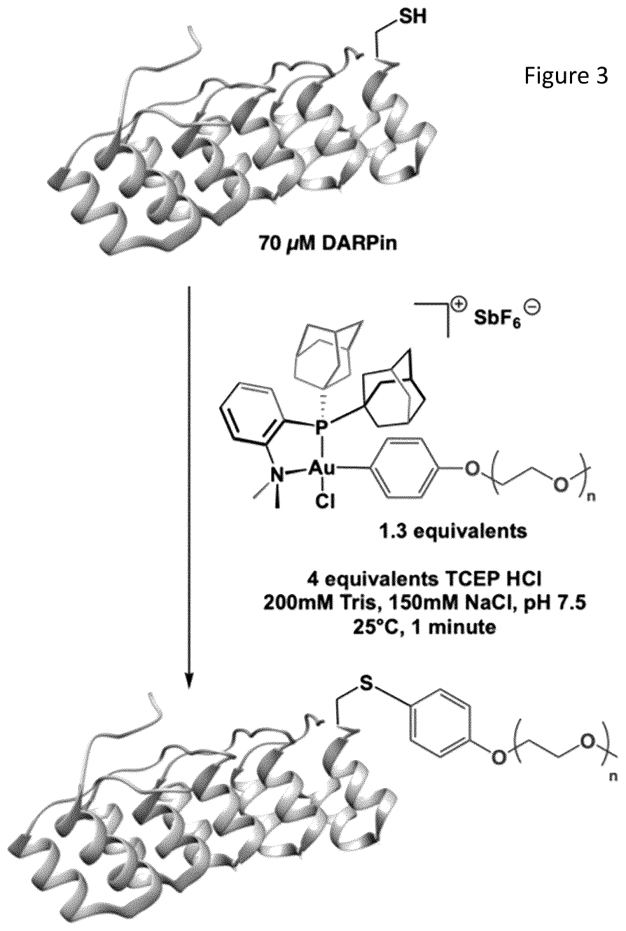

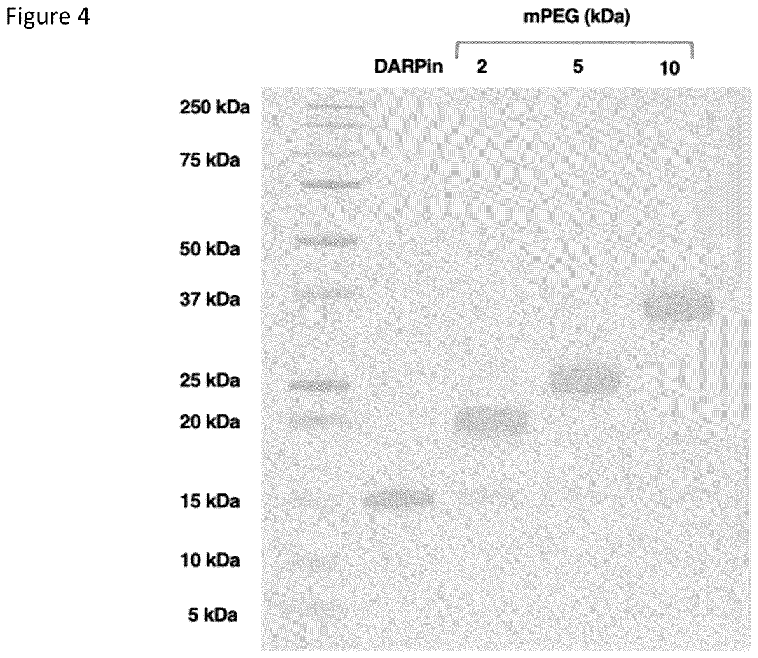

Liquid chromatography-mass spectrometry (LC-MS) experiments were also performed to confirm the absence of degradation or aggregation of the conjugate. The UV and total ion count (TIC) traces showed a peak corresponding to the mPEG-DARPin conjugate with deconvoluted masses corresponding to the mass of mPEG-DARPin for the 2, 5 and 10 kDa conjugates. (FIG. 3c, S24). A peak at a later retention time was also observed corresponding to the Au(I) byproducts produced by this reaction.

Circular Dichroism (CD) Analysis of FPLC Purified DARPin and mPEG-DARPin Conjugate

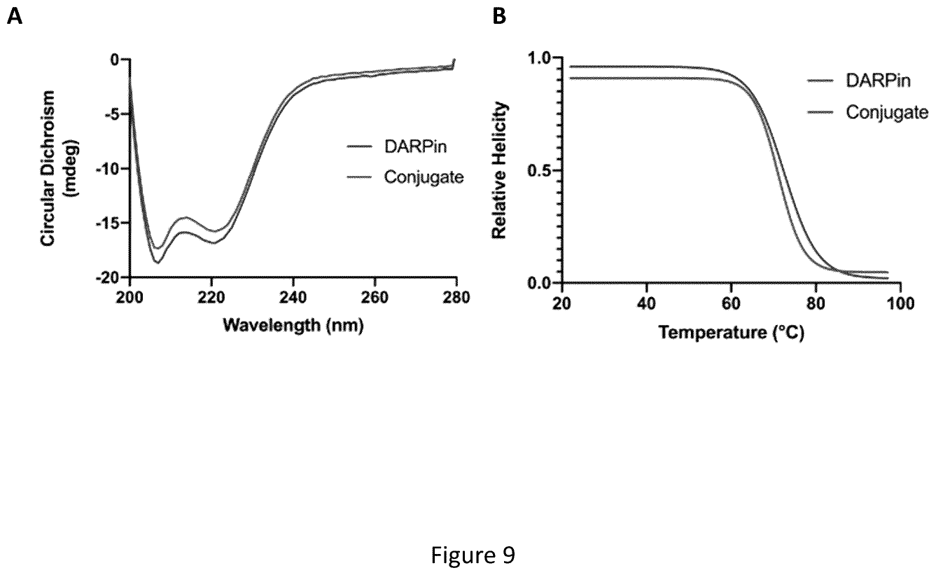

Purified DARPin and 5 kDa mPEG DARPin conjugate were diluted to 7.6 μM in 300 μL total volume of buffer (20 mM Tris, 150 mM NaCl, pH 7.5). CD spectra were taken sweeping from 200-280 nm at 25° C. Thermal denaturation analysis was performed by calculating the relative helicity ((mdeg−mdegₘᵢₙ)/mdegₘₐₓ) at 208 nm from 22-98° C. FIG. 9 a) shows a circular dichroism spectrum of native DARPin and DARPin conjugate at 25° C. showing no significant difference in helicity. FIG. 9 b) shows a CD thermal denaturation curves of native DARPin and DARPin conjugate showing no significant difference in melting temperature between 22-98° C.

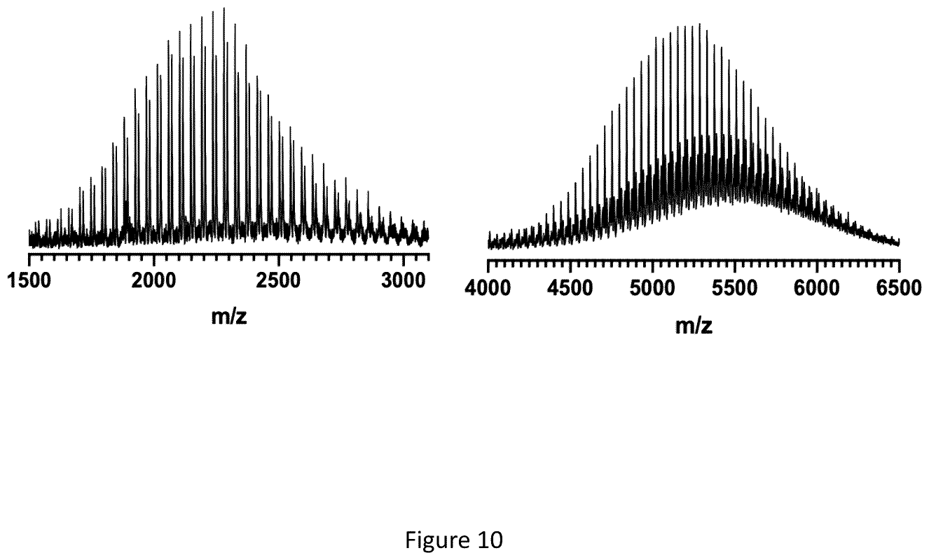

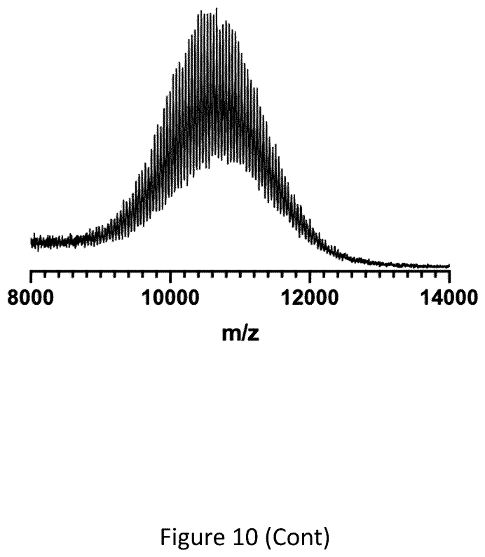

MALDI of mPEG Au(III) Reagents Preparation for analysis: The matrix was prepared by sonicating a solution of 16.5 mg of α-cyano-4-hydroxycinnamic acid (CHCA) in 1 mL ethanol until fully dissolved. The cationizing agent was prepared by sonicating a solution of 6.2 mg of potassium trifluoroacetate (KTFA) in 1 mL ethanol until dissolved. The samples for analysis were prepared at 1 mM in MilliQ water. The three solutions were mixed with a pipette in an Eppendorf in a 5:1:1 matrix:sample:catonizing agent (v/v/v) ratio, and 1 μL was spotted onto a ground steel target plate and allowed to dry before being inserted into the instrument for analysis. FIG. 10 shows the MALDI TOF spectra of 2, 5 and 10 kDa (left to right) mPEG Au(III) reagents.

ICP-OES Analysis of FPLC Purified mPEG-DARPin Conjugate

A 30 μM solution of purified 10 kDa mPEG-DARPin conjugate was quantitatively transferred to a 14 mL centrifuge tube using multiple rinses of MilliQ water, acidified by addition of 0.4 mL HNO3 (FisherChemical Trace Metal Grade Nitric Acid; certified [Au]<0.1 ppb), and then diluted with MilliQ water to 4.0 mL, to give a total sample acidity of ˜10%. This solution was sonicated for 20 minutes and then immediately analyzed via ICP-OES.

The concentration of Au in the sample was quantified using the Au emission of 242.79 nm. An Yttrium internal standard (2 ppm in 10% HNO3) was run simultaneously with all samples and the characteristic Y emission was measured at 371.03 nm. This analysis indicates 98.77%±0.17% (14.91 ppb) efficiency for the removal of Au-containing species by the described purification procedure.

### REFERENCES

(1) Alconcel, S. N. S.; Baas, A. S.; Maynard, H. D. FDA-Approved Poly(Ethylene Glycol)-Protein Conjugate Drugs. Polym. Chem. 2011, 2 (7), 1442-1448. https://doi.org/10.1039/C1PY00034A.
(2) FDA Approved PEGylated Drugs Up To 2022|Biopharma PEG. https://www.biochempeg.com/article/58.html (accessed 2022-05-20).
(3) Gupta, V.; Bhavanasi, S.; Quadir, M.; Singh, K.; Ghosh, G.; Vasamreddy, K.; Ghosh, A.; Siahaan, T. J.; Banerjee, S.; Banerjee, S. K. Protein PEGylation for Cancer Therapy: Bench to Bedside. J. Cell Commun. Signal. 2019, 13 (3), 319-330. https://doi.org/10.1007/s12079-018-0492-0.
(4) Harris, J. M.; Chess, R. B. Effect of Pegylation on Pharmaceuticals. Nat. Rev. Drug Discov. 2003, 2 (3), 214-221. https://doi.org/10.1038/nrd1033.
(5) Lawrence, P. B.; Price, J. L. How PEGylation Influences Protein Conformational Stability. Curr. Opin. Chem. Biol. 2016, 34, 88-94. https://doi.org/10.1016/j.cbpa.2016.08.006.
(6) Yang, C.; Lu, D.; Liu, Z. How PEGylation Enhances the Stability and Potency of Insulin: A Molecular Dynamics Simulation. Biochemistry 2011, 50 (13), 2585-2593. https://doi.org/10.1021/bi101926u.
(7) Diwan, M.; Park, T. G. Pegylation Enhances Protein Stability during Encapsulation in PLGA Microspheres. J. Controlled Release 2001, 73 (2), 233-244. https://doi.org/10.1016/S0168-3659(01)00292-9.
(8) White, C. J.; Bode, J. W. PEGylation and Dimerization of Expressed Proteins under Near Equimolar Conditions with Potassium 2-Pyridyl Acyltrifluoroborates. ACS Cent. Sci. 2018, 4 (2), 197-206. https://doi.org/10.1021/acscentsci.7b00432.
(9) Swierczynski, M. J.; Ball, Z. T. One-Step Protein-Polymer Conjugates from Boronic-Acid-Functionalized Polymers. Bioconjug. Chem. 2020, 31 (11), 2494-2498. https://doi.org/10.1021/acs.bioconjchem.0c00516.
(10) Deiters, A.; Cropp, T. A.; Summerer, D.; Mukherji, M.; Schultz, P. G. Site-Specific PEGylation of Proteins Containing Unnatural Amino Acids. Bioorg. Med. Chem. Lett. 2004, 14 (23), 5743-5745. https://doi.org/10.1016/j.bmcl.2004.09.059.
(11) Mantovani, G.; Lecolley, F.; Tao, L.; Haddleton, D. M.; Clerx, J.; Cornelissen, J. J. L. M.; Velonia, K. Design and Synthesis of N-Maleimido-Functionalized Hydrophilic Polymers via Copper-Mediated Living Radical Polymerization: A Suitable Alternative to PEGylation Chemistry. J. Am. Chem. Soc. 2005, 127 (9), 2966-2973. https://doi.org/10.1021/ja0430999.
(12) Stenzel, M. H. Bioconjugation Using Thiols: Old Chemistry Rediscovered to Connect Polymers with Nature's Building Blocks. ACS Macro Lett. 2013, 2 (1), 14-18. https://doi.org/10.1021/mz3005814.
(13) Turecek, P. L.; Bossard, M. J.; Schoetens, F.; Ivens, I. A. PEGylation of Biopharmaceuticals: A Review of Chemistry and Nonclinical Safety Information of Approved Drugs. J. Pharm. Sci. 2016, 105 (2), 460-475. https://doi.org/10.1016/j.xphs.2015.11.015.
(14) Belen, L. H.; Rangel-Yagui, C. de O.; Beltrán Lissabet, J. F.; Effer, B.; Lee-Estevez, M.; Pessoa, A.; Castillo, R. L.; Farias, J. G. From Synthesis to Characterization of Site-Selective PEGylated Proteins. Front. Pharmacol. 2019, 10.
(15) Dumas, A.; Spicer, C. D.; Gao, Z.; Takehana, T.; Lin, Y. A.; Yasukohchi, T.; Davis, B. G. Self-Liganded Suzuki-Miyaura Coupling for Site-Selective Protein PEGylation. Angew. Chem. Int. Ed. 2013, 52 (14), 3916-3921. https://doi.org/10.1002/anie.201208626.
(16) Ko, H.-M.; Deng, J.-R.; Cui, J.-F.; Kung, K. K.-Y.; Leung, Y.-C.; Wong, M.-K. Selective Modification of Alkyne-Linked Peptides and Proteins by Cyclometalated Gold(III) (C\hat{ }N) Complex-Mediated Alkynylation. Bioorg. Med. Chem. 2020, 28 (7), 115375. https://doi.org/10.1016/j.bmc.2020.115375.
(17) Wang, M.; Svatunek, D.; Rohlfing, K.; Liu, Y.; Wang, H.; Giglio, B.; Yuan, H.; Wu, Z.; Li, Z.; Fox, J. Conformationally Strained Trans-Cyclooctene (STCO) Enables the Rapid Construction of 18 F-PET Probes via Tetrazine Ligation. Theranostics 2016, 6 (6), 887-895. https://doi.org/10.7150/thno.14742.
(18) Laserna, V.; Abegg, D.; Afonso, C. F.; Martin, E. M.; Adibekian, A.; Ravn, P.; Corzana, F.; Bernardes, G. J. L. Dichloro Butenediamides as Irreversible Site-Selective Protein Conjugation Reagent. Angew. Chem. Int. Ed. 2021, 60 (44), 23750-23755. https://doi.org/10.1002/anie.202108791.
(19) Konc, J.; Brown, L.; Whiten, D. R.; Zuo, Y.; Ravn, P.; Klenerman, D.; Bernardes, G. J. L. A Platform for Site-Specific DNA-Antibody Bioconjugation by Using Benzoylacrylic-Labelled Oligonucleotides. Angew. Chem. Int. Ed. 2021, 60 (49), 25905-25913. https://doi.org/10.1002/anie.202109713.
(20) Ohata, J.; Krishnamoorthy, L.; Gonzalez, M. A.; Xiao, T.; Iovan, D. A.; Toste, F. D.; Miller, E. W.; Chang, C. J. An Activity-Based Methionine Bioconjugation Approach To Developing Proximity-Activated Imaging Reporters. ACS Cent. Sci. 2020, 6 (1), 32-40. https://doi.org/10.1021/acscentsci.9b01038.
(21) Cheng, W.-M.; Lu, X.; Shi, J.; Liu, L. Selective Modification of Natural Nucleophilic Residues in Peptides and Proteins Using Arylpalladium Complexes. Org. Chem. Front. 2018, 5 (21), 3186-3193. https://doi.org/10.1039/C8QO00765A.
(22) Gunnoo, S. B.; Madder, A. Chemical Protein Modification through Cysteine. ChemBioChem 2016, 17 (7), 529-553. https://doi.org/10.1002/cbic.201500667.
(23) Ochtrop, P.; Hackenberger, C. P. R. Recent Advances of Thiol-Selective Bioconjugation Reactions. Curr. Opin. Chem. Biol. 2020, 58, 28-36. https://doi.org/10.1016/j.cbpa.2020.04.017.
(24) You, J.; Zhang, J.; Wang, J.; Jin, M. Cysteine-Based Coupling: Challenges and Solutions. Bioconjug. Chem. 2021, 32 (8), 1525-1534. https://doi.org/10.1021/acs.bioconjchem.1c00213.
(25) Lyon, R. P.; Setter, J. R.; Bovee, T. D.; Doronina, S. O.; Hunter, J. H.; Anderson, M. E.; Balasubramanian, C. L.; Duniho, S. M.; Leiske, C. I.; Li, F.; et al. Self-Hydrolyzing Maleimides Improve the Stability and Pharmacological Properties of Antibody-Drug Conjugates. Nat. Biotechnol. 2014, 32 (10), 1059-1062. https://doi.org/10.1038/nbt.2968.
(26) Shen, B.-Q.; Xu, K.; Liu, L.; Raab, H.; Bhakta, S.; Kenrick, M.; Parsons-Reponte, K. L.; Tien, J.; Yu, S.-F.; Mai, E.; et al. Conjugation Site Modulates the in Vivo Stability and Therapeutic Activity of Antibody-Drug Conjugates. Nat. Biotechnol. 2012, 30 (2), 184-189. https://doi.org/10.1038/nbt.2108.
(27) Zhang, C.; Liu, Y.; Feng, C.; Wang, Q.; Shi, H.; Zhao, D.; Yu, R.; Su, Z. Loss of PEG Chain in Routine SDS-PAGE Analysis of PEG-Maleimide Modified Protein. ELECTROPHORESIS 2015, 36 (2), 371-374. https://doi.org/10.1002/elps.201400373.
(28) Hermanson, G. T., Bioconjugate Techniques, 3rd ed. Academic Press: New York, 1996.
(29) Altinbasak, I.; Arslan, M.; Sanyal, R.; Sanyal, A. Pyridyl Disulfide-Based Thiol-Disulfide Exchange Reaction: Shaping the Design of Redox-Responsive Polymeric Materials. Polym. Chem. 2020, 11 (48), 7603-7624. https://doi.org/10.1039/DOPY01215G.
(30) Shakya, A. K.; Sami, H.; Srivastava, A.; Kumar, A. Stability of Responsive Polymer-Protein Bioconjugates. Prog. Polym. Sci. 2010, 35 (4), 459-486. https://doi.org/10.1016/j.progpolymsci.2010.01.003.
(31) Masters, J. C.; Nickens, D. J.; Xuan, D.; Shazer, R. L.; Amantea, M. Clinical Toxicity of Antibody Drug Conjugates: A Meta-Analysis of Payloads. Invest. New Drugs 2018, 36 (1), 121-135. https://doi.org/10.1007/s10637-017-0520-6.
(32) Behrens, C. R.; Ha, E. H.; Chinn, L. L.; Bowers, S.; Probst, G.; Fitch-Bruhns, M.; Monteon, J.; Valdiosera, A.; Bermudez, A.; Liao-Chan, S.; et al. Antibody-Drug Conjugates (ADCs) Derived from Interchain Cysteine Cross-Linking Demonstrate Improved Homogeneity and Other Pharmacological Properties over Conventional Heterogeneous ADCs. Mol. Pharm. 2015, 12 (11), 3986-3998. https://doi.org/10.1021/acs.molpharmaceut.5b00432.
(33) Baldwin, A. D.; Kiick, K. L. Tunable Degradation of Maleimide-Thiol Adducts in Reducing Environments. Bioconjug. Chem. 2011, 22 (10), 1946-1953. https://doi.org/10.1021/bc200148v.
(34) Vinogradova, E. V.; Zhang, C.; Spokoyny, A. M.; Pentelute, B. L.; Buchwald, S. L. Organometallic Palladium Reagents for Cysteine Bioconjugation. Nature 2015, 526 (7575), 687-691. https://doi.org/10.1038/nature15739.
(35) Lobba, M. J.; Fellmann, C.; Marmelstein, A. M.; Maza, J. C.; Kissman, E. N.; Robinson, S. A.; Staahl, B. T.; Urnes, C.; Lew, R. J.; Mogilevsky, C. S.; et al. Site-Specific Bioconjugation through Enzyme-Catalyzed Tyrosine-Cysteine Bond Formation. ACS Cent. Sci. 2020, 6 (9), 1564-1571. https://doi.org/10.1021/acscentsci.0c00940.
(36) Messina, M. S.; Stauber, J. M.; Waddington, M. A.; Rheingold, A. L.; Maynard, H. D.; Spokoyny, A. M. Organometallic Gold(III) Reagents for Cysteine Arylation. J. Am. Chem. Soc. 2018, 140 (23), 7065-7069. https://doi.org/10.1021/jacs.8b04115.
(37) Zhang, C.; Vinogradova, E. V.; Spokoyny, A. M.; Buchwald, S. L.; Pentelute, B. L. Arylation Chemistry for Bioconjugation. Angew. Chem. Int. Ed. 2019, 58 (15), 4810-4839. https://doi.org/10.1002/anie.201806009.
(38) Stauber, J. M.; Qian, E. A.; Han, Y.; Rheingold, A. L.; Král, P.; Fujita, D.; Spokoyny, A. M. An Organometallic Strategy for Assembling Atomically Precise Hybrid Nanomaterials. J. Am. Chem. Soc. 2020, 142 (1), 327-334. https://doi.org/10.1021/jacs.9b10770.
(39) Joost, M.; Amgoune, A.; Bourissou, D. Reactivity of Gold Complexes towards Elementary Organometallic Reactions. Angew. Chem. Int. Ed. 2015, 54 (50), 15022-15045. https://doi.org/10.1002/anie.201506271.
(40) Joost, M.; Zeineddine, A.; Estevez, L.; Mallet-Ladeira, S.; Miqueu, K.; Amgoune, A.; Bourissou, D. Facile Oxidative Addition of Aryl Iodides to Gold(I) by Ligand Design: Bending Turns on Reactivity. J. Am. Chem. Soc. 2014, 136 (42), 14654-14657. https://doi.org/10.1021/ja506978c.
(41) Zeineddine, A.; Estevez, L.; Mallet-Ladeira, S.; Miqueu, K.; Amgoune, A.; Bourissou, D. Rational Development of Catalytic Au(I)/Au(III) Arylation Involving Mild Oxidative Addition of Aryl Halides. Nat. Commun. 2017, 8 (1), 565. https://doi.org/10.1038/s41467-017-00672-8.
(42) Rodriguez, J.; Zeineddine, A.; Carrizo, E. D. S.; Miqueu, K.; Saffon-Merceron, N.; Amgoune, A.; Bourissou, D. Catalytic Au(I)/Au(III) Arylation with the Hemilabile MeDalphos Ligand: Unusual Selectivity for Electron-Rich Iodoarenes and Efficient Application to Indoles. Chem. Sci. 2019, 10 (30), 7183-7192. https://doi.org/10.1039/C9SC01954E.
(43) De, P.; Li, M.; Gondi, S. R.; Sumerlin, B. S. Temperature-Regulated Activity of Responsive Polymer-Protein Conjugates Prepared by Grafting-from via RAFT Polymerization. J. Am. Chem. Soc. 2008, 130 (34), 11288-11289. https://doi.org/10.1021/ja804495v.
(44) Tao, L.; Kaddis, C. S.; Loo, R. R. O.; Grover, G. N.; Loo, J. A.; Maynard, H. D. Synthesis of Maleimide-End-Functionalized Star Polymers and Multimeric Protein-Polymer Conjugates. Macromolecules 2009, 42 (21), 8028-8033. https://doi.org/10.1021/ma901540p.
(45) Vinogradova, E. V. Organometallic Chemical Biology: An Organometallic Approach to Bioconjugation. Pure Appl. Chem. 2017, 89 (11), 1619-1640. https://doi.org/10.1515/pac-2017-0207.
(46) Malins, L. R. Peptide Modification and Cyclization via Transition-Metal Catalysis. Curr. Opin. Chem. Biol. 2018, 46, 25-32. https://doi.org/10.1016/j.cbpa.2018.03.019.
(47) Rodriguez, J.; Martinez-Calvo, M. Transition-Metal-Mediated Modification of Biomolecules. Chem.-Eur. J. 2020, 26 (44), 9792-9813. https://doi.org/10.1002/chem.202001287.
(48) Waddington, M. A.; Zheng, X.; Stauber, J. M.; Hakim Moully, E.; Montgomery, H. R.; Saleh, L. M. A.; Král, P.; Spokoyny, A. M. An Organometallic Strategy for Cysteine Borylation. J. Am. Chem. Soc. 2021, 143 (23), 8661-8668. https://doi.org/10.1021/jacs.1c02206.
(49) Jbara, M. Transition Metal Catalyzed Site-Selective Cysteine Diversification of Proteins. Pure Appl. Chem. 2021, 93 (2), 169-186. https://doi.org/10.1515/pac-2020-0504.
(50) Ohata, J.; T. Ball, Z. Rhodium at the Chemistry-Biology Interface. Dalton Trans. 2018, 47 (42), 14855-14860. https://doi.org/10.1039/C8DT03032D.
(51) Zhao, Z.; Shimon, D.; Metanis, N. Chemoselective Copper-Mediated Modification of Selenocysteines in Peptides and Proteins. J. Am. Chem. Soc. 2021, 143 (32), 12817-12824. https://doi.org/10.1021/jacs.1c06101.
(52) Lin, X.; Haimov, E.; Redko, B.; Vigalok, A. Selective Stepwise Arylation of Unprotected Peptides by PtIV Complexes. Angew. Chem. Int. Ed. n/a (n/a), e202205368. https://doi.org/10.1002/anie.202205368.
(53) Tilden, J. A. R.; Lubben, A. T.; Reeksting, S. B.; Kociok-Kohn, G.; Frost, C. G. Pd(II)-Mediated C—H Activation for Cysteine Bioconjugation. Chem.-Eur. J. 2022, 28 (11), e202104385. https://doi.org/10.1002/chem.202104385.
(54) Yang, P.; Wang, X.; Li, B.; Yang, Y.; Yue, J.; Suo, Y.; Tong, H.; He, G.; Lu, X.; Chen, G. Streamlined Construction of Peptide Macrocycles via Palladium-Catalyzed Intramolecular S-Arylation in Solution and on DNA. Chem. Sci. 2021, 12 (16), 5804-5810. https://doi.org/10.1039/D1SC00789K.
(55) Gukathasan, S.; Parkin, S.; Black, E. P.; Awuah, S. G. Tuning Cyclometalated Gold(III) for Cysteine Arylation and Ligand-Directed Bioconjugation. Inorg. Chem. 2021, 60 (19), 14582-14593. https://doi.org/10.1021/acs.inorgchem.1c01517.
(56) Wang, W.; Lorion, M. M.; Shah, J.; Kapdi, A. R.; Ackermann, L. Late-Stage Peptide Diversification by Position-Selective C—H Activation. Angew. Chem. Int. Ed. 2018, 57 (45), 14700-14717. https://doi.org/10.1002/anie.201806250.

### From Examples:

(1) Lundgren, R. J.; Sappong-Kumankumah, A.; Stradiotto, M. A Highly Versatile Catalyst System for the Cross-Coupling of Aryl Chlorides and Amines. Chem.-Eur. J. 2010, 16 (6), 1983-1991. https://doi.org/10.1002/chem.200902316.
(2) Hesp, K. D.; Stradiotto, M. Stereo- and Regioselective Gold-Catalyzed Hydroamination of Internal Alkynes with Dialkylamines. J. Am. Chem. Soc. 2010, 132 (51), 18026-18029. https://doi.org/10.1021/ja109192w.
(3) Chen, Y.; Chen, J.; Zhang, N.; Ye, L.; Zhang, X.-J.; Yan, M. A New Synthesis of Indoles via Intramolecular Cyclization of o-Alkynyl N,N-Dialkylanilines Promoted by KOt-Bu/DMSO. Tetrahedron Lett. 2015, 56 (2), 478-481. https://doi.org/10.1016/j.tetlet.2014.12.002.
(4) Messina, M. S.; Stauber, J. M.; Waddington, M. A.; Rheingold, A. L.; Maynard, H. D.; Spokoyny, A. M. Organometallic Gold(III) Reagents for Cysteine Arylation. J. Am. Chem. Soc. 2018, 140 (23), 7065-7069. https://doi.org/10.1021/jacs.8b04115.
(5) Vinogradova, E. V.; Zhang, C.; Spokoyny, A. M.; Pentelute, B. L.; Buchwald, S. L. Organometallic Palladium Reagents for Cysteine Bioconjugation. Nature 2015, 526 (7575), 687-691. https://doi.org/10.1038/nature15739.
(6) Wingfield, P. T. N-Terminal Methionine Processing. Curr. Protoc. Protein Sci. 2017, 88 (1), 6.14.1-6.14.3. https://doi.org/10.1002/cpps.29.

We claim:

1. A composition comprising a polypeptide having at least one cysteine residue and a gold(III) aryl complex, the gold(III) aryl complex comprising a polyethylene glycol group having an average molecular weight greater than 1 kDa attached to the aryl group.

2. The composition of claim 1, wherein the polyethylene glycol group has an average molecular weight greater than 2 kDa.

3. The composition of claim 1, wherein the composition has a molar ratio of the gold(III) aryl complex to the polypeptide of less than 5.

4. The composition of claim 3, wherein the molar ratio of the gold(III) aryl complex to the polypeptide is between 1 and 2.

5. The composition of claim 1, wherein the polypeptide has a molecular weight greater than 10 kDa.

6. The composition of claim 1, wherein the gold(III) aryl complex is

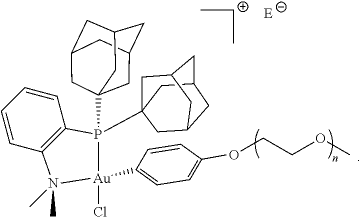

7. The composition of claim 1 further comprising one or more of a reducing agent, a buffer, a salt, and solvent.

8. The composition of claim 1, wherein the composition has a pH of between 5.5 and 7.5.

9. The composition of claim 1, wherein the composition comprises more than 50 μM of the polypeptide.

10. The composition of claim 1 further comprising a cysteine-aryl conjugated polypeptide.

11. A method of preparing a cysteine-aryl conjugated polypeptide, the method comprising contacting the gold(III) aryl complex and the polypeptide according to the composition of claim 1 under conditions sufficient to prepare the cysteine-aryl conjugated polypeptide.

12. The method of claim 11, wherein the gold(III) aryl complex and the polypeptide are contacted at a temperature less than 30° C.

13. The method of claim 11, the gold(III) aryl complex and the polypeptide are contacted for a time less than 5 minutes.

14. The method of claim 11, wherein the conditions are sufficient to convert at least 90% of the polypeptide to the S-aryl conjugated polypeptide.

15. The method of claim 11 further comprising purifying the cysteine-aryl conjugated polypeptide.

16. The method of claim 15, wherein the cysteine-aryl conjugated polypeptide is purified by size exclusion-fast protein liquid chromatography, dialysis, or spin filtration.

17. A method of preparing a gold(III) aryl complex comprising heating a composition comprising a gold(I) precursor, a halide scavenger, and a pegylated aryl iodide comprising a polyethylene glycol group having an average molecular weight greater than 1 kDa attached to an aryl group.

18. The method of claim 17, wherein the composition comprises an excess of both the gold (I) precursor and halide scavenger to the pegylated aryl iodide.

19. The method of claim 17 further comprising purifying the gold(III) aryl complex.

20. The method of claim 17, wherein the gold(I) precursor is

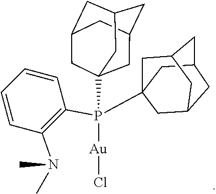

21. The method of claim 17, wherein the halide scavenger is silver hexafluoroantimonate(V) (AgSbF₆).
